(19) United States
(12) Patent Application Publication
(10) Pub. No.: US 2001/0034534 A1
(43) Pub. Date: Oct. 25, 2001

(54) Apparatus and method for making incisions for hair grafts

(76) Inventor: James Transue, Bridgewater, NJ (US)

Correspondence Address: Stroock & Stroock & Lavan LLP, 180 Maiden Lane, New York, NY, 10038, US

(21) Appl. No.: 09/771,216

(22) Filed: Jan. 26, 2001

Related U.S. Application Data
(60) Provisional application No. 60/178,150, filed on Jan. 26, 2000.
(60) Provisional application No. 60/237,526, filed on Oct. 4, 2000.

(51) Int. Cl. A61B 17/34

(52) U.S. Cl. 606/186; 606/187

(57) ABSTRACT

An apparatus is provided for forming hair graft incisions in a hair transplantation procedure. The apparatus includes an elongated handle extending along a longitudinal axis, and a plurality of blades mounted to and extending from a distal end of the handle. The blades are arranged along a single blade axis that is disposed oblique to, preferably perpendicularly to, the longitudinal axis of the handle.

### CROSS-REFERENCE TO RELATED APPLICATIONS

This Application claims priority of U.S. Provisional Application No. 60/178,150, filed Jan. 26, 2000, and claims priority of U.S. Provisional Application No. 60/237,526, filed Oct. 4, 2000.

### BACKGROUND OF THE INVENTION

A typical method of hair transplantation requires transplantation of hair from one location on a person's body to a second desired location, such as a bald spot on a person's scalp. Specifically, the process involves harvesting strips of hair-laden skin, which are commonly referred to as donor strips, from the location where the hair is to be taken. An apparatus, such as that shown in U.S. Pat. No. 5,989,273 to Arnold, is used to incise the person's flesh and harvest the donor strips. As disclosed therein, the harvesting apparatus uses scalpel blades which are inserted into a person's skin and urged to form extended, continuous incisions which define the donor strips.

Thereafter, the donor strips are divided into a plurality of hair grafts, with each graft containing a small number of hairs (e.g., two to six hairs, commonly referred to as a minigraft), or a single hair (referred to as a micrograft). A surgeon then proceeds to make discrete hair graft incisions in the area desired for hair transplantation. During this process, the doctor must make incisions of about 5.5 millimeters deep to ensure sufficient depth to properly receive the hair graft while preventing damage to the skull bone and deep vascularity. Furthermore, because a significant number of hairs are being transplanted at once to, in effect, recreate a healthy hair region, adjacent incisions must be precisely spaced from each other so as not to interfere with each other, yet provide sufficient density of transplanted hair for overall esthetic appearance. This spacing has generally been developed at about 3.0 millimeters between adjacent incisions. Accordingly, making one incision at a time, the process is exacting, requiring detailed and accurate mapping of the scalp as well as the ability to follow the mapped layout of incisions.

In addition, it is desired to make the incisions with different depths and angular orientations to achieve a natural appearance for transplanted hair. With the incisions being made, individual hair grafts (minigrafts or micrografts) are inserted into each of the incisions.

In the past, surgeons have used apparatuses which make single incisions in preparing incisions for the hair grafts, such as ophthalmic blades or other instruments, such as the apparatus disclosed in U.S. Pat. No. 5,693,064 to Arnold. Since a high number of incisions are typically required in this procedure, at least one apparatus has been developed in the prior art which is capable of simultaneously forming a multiplicity of incisions, thereby reducing the time and effort required in the transplantation procedure. To wit, U.S. Pat. No. 5,858,019 to Ashraf discloses a graft site cutter which simultaneously makes a plurality of incisions arranged in a fixed matrix. The Ashraf apparatus utilizes a plurality of knives which are rigidly fixed to a member, such member being disposed above a resilient sponge. Upon compressing the rigid member into the sponge, the knives extend through the sponge to make the incisions.

There is, however, a drawback with the Ashraf apparatus. The apparatus makes a very high number of incisions over a relatively large area, and, as such the Ashraf apparatus does not allow for control of individual, or for that matter, a limited number of incisions, thus reducing the ability to make incisions of different depth and angles relative to the scalp. Because of the large area of and limited control over the incisions, the ability to control natural-looking placement and angular disposition of transplanted hair is limited with the Ashraf apparatus.

It is an object of the subject invention to provide an apparatus and method for making hair graft incisions.

### SUMMARY OF THE INVENTION

To overcome the deficiencies of the prior art, an apparatus and method is provided for simultaneously preparing a limited number, preferably on the order of three, hair graft incisions. With the subject invention, a plurality of incisions can be simultaneously made with a surgeon having the ability to adapt and make hair graft incisions to naturally follow a person's scalp.

The apparatus of the subject invention includes a handle having a flat distal end and a plurality of, preferably three, blades being mounted thereon. The blades are of the type known in the art to create single punctures, such as “stab blades” and “spear blades”. The handle is elongated and formed to extend along a longitudinal axis. In addition, the blades are aligned on a single blade axis which is oblique to, preferably perpendicular to, the longitudinal axis of the handle.

The blades are evenly-spaced along the blade axis and uniformly extend the same distance from the distal end of the handle. With this arrangement, a surgeon has good angular control over the apparatus, in forming incisions which are not only evenly spaced, but also have the same incision angle. It is preferred that the extent of the blades be limited, so that the distal end of the handle prevents excessive insertion of the blades into a person's flesh, thereby avoiding blunting of one or more of the blades against bone (e.g. a skull) and/or causing damage to deep vascularity.

These and other features of the invention will be better understood through a study of the following detailed description and accompanying drawings.

### BRIEF DESCRIPTION OF THE DRAWINGS

For a fuller understanding of the invention, reference is made to the following description taken in connection with the accompanying drawings, in which:

FIG. 1 is a top view of an apparatus of the subject invention;

FIG. 2 is a side view of the apparatus;

FIG. 3 is a perspective view of a handle of the apparatus;

FIG. 4 is an enlarged end view of the handle;

FIG. 4A is an enlarged end view of an alternative embodiment of a blind aperture formed in the handle;

FIG. 5 is a top view of the handle;

FIG. 6 is a top view of one type of blade usable with the apparatus;

FIG. 7 is a top view of a second type of blade usable with the apparatus;

FIG. 8 is a schematic of the apparatus being inserted into the flesh of a patient to form a hair graft incision therein;

FIG. 9 is a top view of the apparatus with the blade shown in FIG. 7;

FIG. 10 is a side view of the apparatus as shown in FIG. 9;

FIG. 11 is a partial view of the apparatus having the blades arranged in a parallel configuration;

FIG. 12 is a perspective view of the apparatus shown in FIG. 1; and FIG. 13 is a perspective view of the apparatus partially shown in FIG. 11.

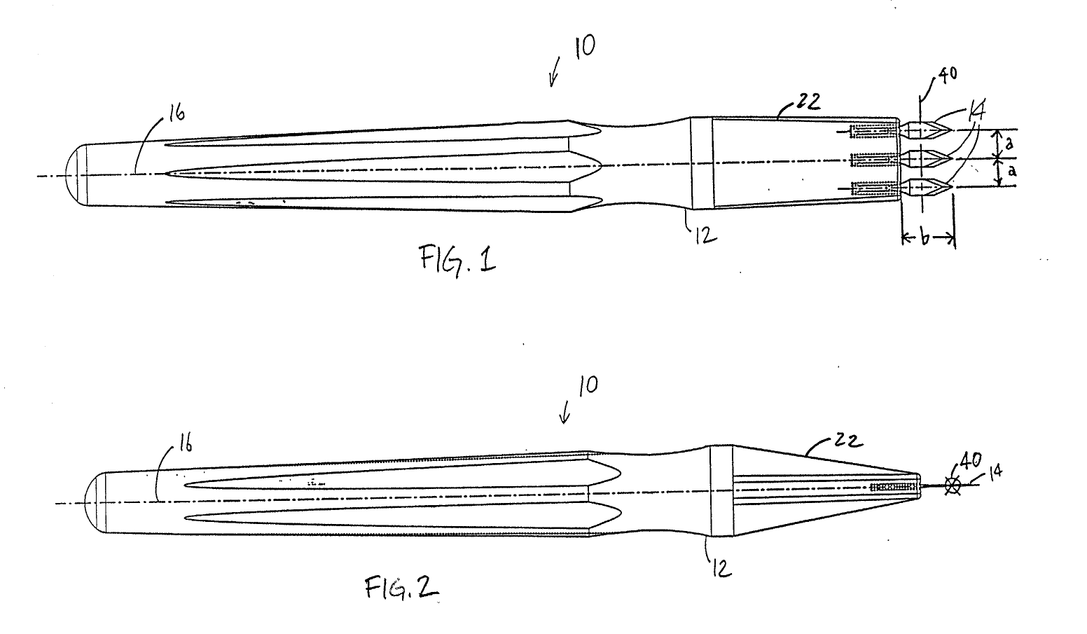

### DETAILED DESCRIPTION OF THE INVENTION

Reference is made to the figures which generally show an apparatus for making hair graft incisions, the apparatus being designated with the reference numeral 10. The apparatus 10 includes a handle 12 and a plurality of, preferably three, blades 14.

With reference to FIGS. 3, 4 and 5, the handle 12 is elongated and formed generally along a longitudinal axis 16. A proximal end 18 of the handle 12 is spaced apart from a distal end 20 along the longitudinal axis 16. Preferably, the distal end 20 is formed to be generally flat in a plane perpendicular to the longitudinal axis 16. A tapered portion 22 extends proximally from the distal end 20, such tapered portion 22 providing a surgeon a less hindered view of the blades 14 as viewed in the direction generally parallel to the longitudinal axis 16. As is readily appreciated, during use of the apparatus 10, a surgeon will be generally viewing the point of incisions from above the apparatus 10 and in a direction generally parallel to the longitudinal axis 16. Between the tapered portion 22 and the proximal end 18, the handle 12 is formed with any grip configuration which is comfortable to handle and allows for easily manipulation thereof. By of way of non-limiting example, ribs 24 are formed on the handle 12 which are circumferentially spaced-apart and sufficiently separated by channels 25, to provide a comfortable grip. Also, the ribs 24 diverge distally from the proximal end 18 and terminate in a concave waist 26, shaped to be gripped by the ends of the fingers of a surgeon. With this configuration, the handle 12, and thus the apparatus 10, can be held and handled much like an ordinary pencil. Preferably, the handle 12 is unitarily formed of, and is comprised of, a thermoplastic material.

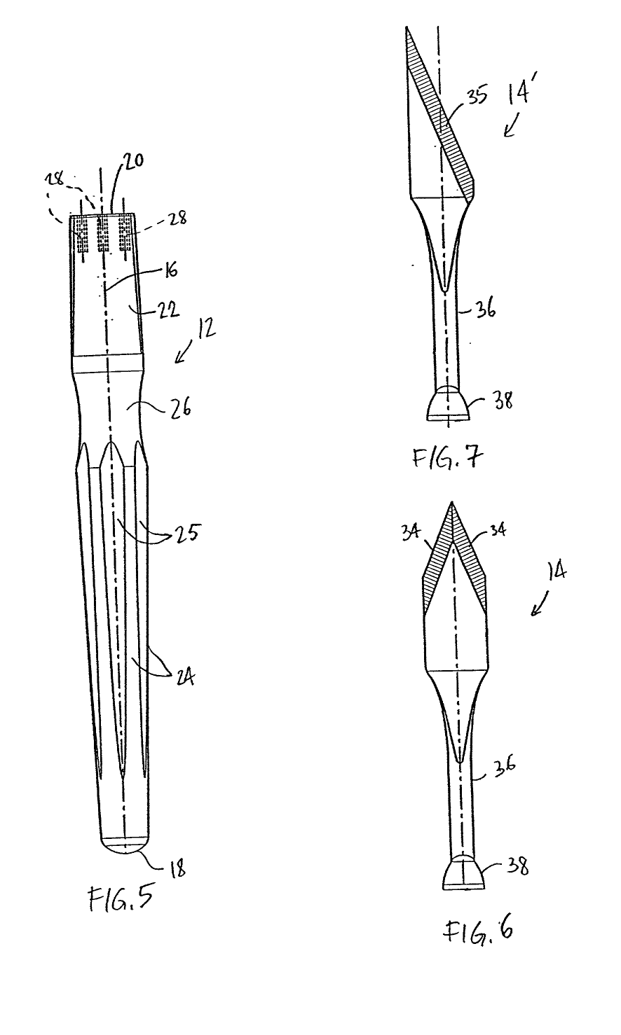

Blind apertures 28 extend proximally through the distal end 20. The shape and number of the blind apertures 28 correspond to the shape and number of the blades 14. The blades 14 may be mounted to the handle 12 using any technique known to those skilled in the art. For example, the blades 14 may be forced fit into the blind apertures 28. To facilitate such force fitting, the blind apertures 28 are each cross-sectionally formed as best shown in FIG. 4, with a generally rectangular portion 30 and a circular portion 32 being superimposed thereon. Depending on the arrangement of the blades 14 (being parallel or perpendicular as discussed below), the rectangular portions 30 may be horizontally aligned, as shown in solid lines in FIG. 4, or vertically aligned, as shown in phantom lines in FIG. 4. In addition, as shown in FIG. 4A, to provide maximum versatility with a single handle design, rectangular portions 30 may be disposed both horizontally and vertically, thereby providing automatic alignment of blades 14 in either a parallel or coplanar arrangement.

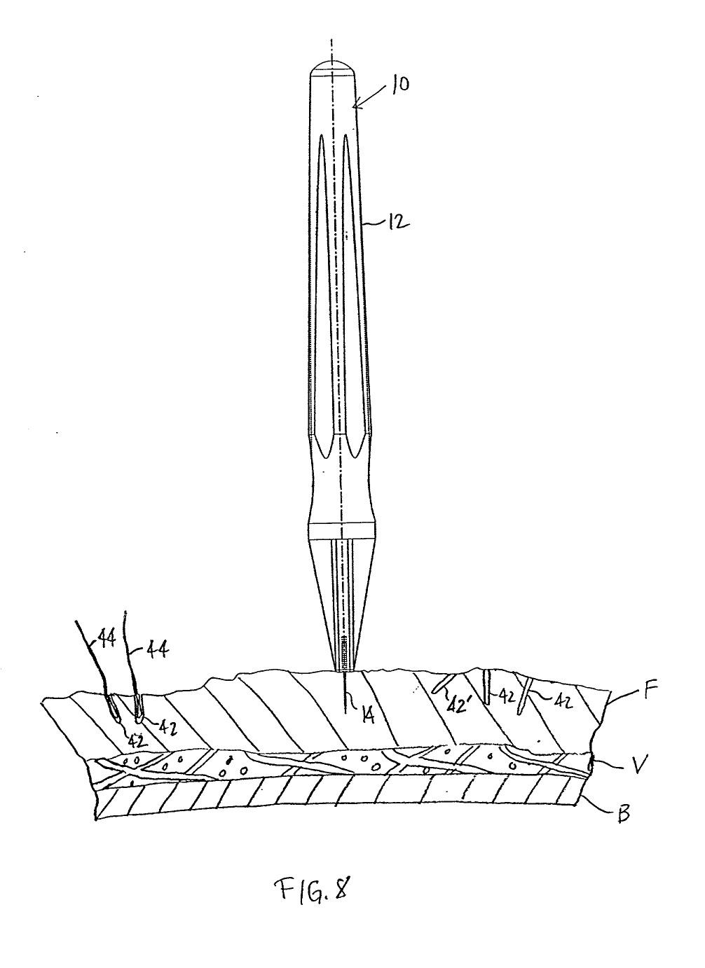

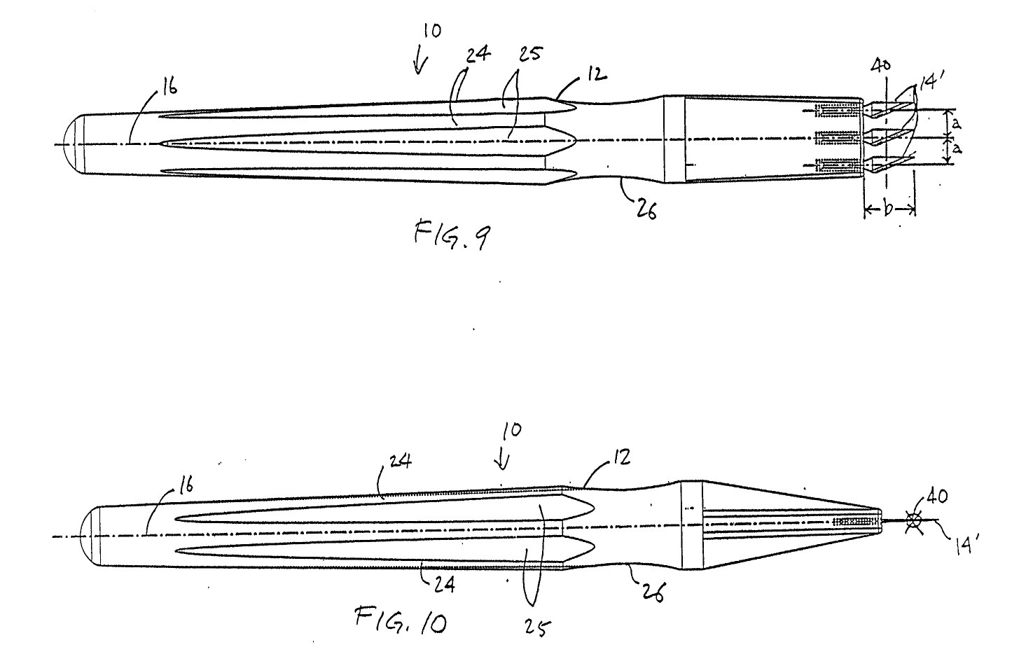

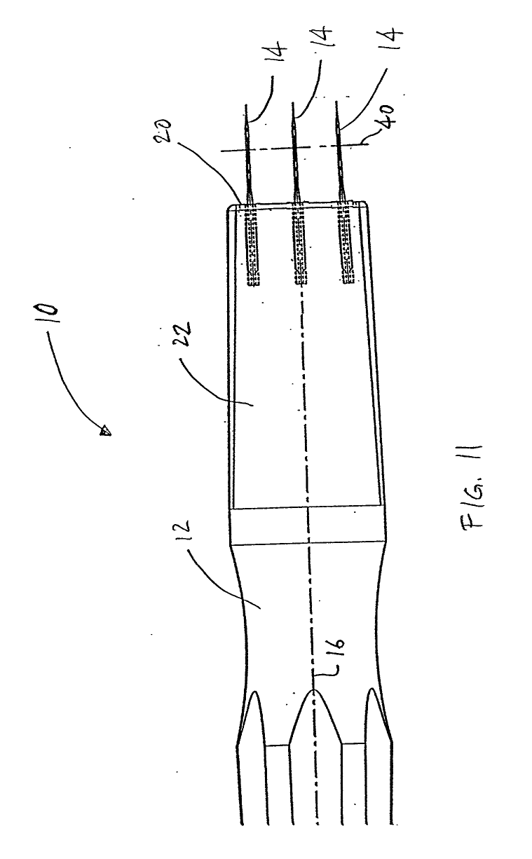

The blades 14 are of the type known in the art to create single punctures. For example, the blades 14 are shown to be of the type known in the art as spear blades. As best shown in FIG. 6, the blades 14 have a generally spear-shape, with two cutting edges 34 merging to define a chevron shape. Alternatively, as shown in FIGS. 9 and 10, the apparatus 10 may have blades of a different shape, such as the blades 14′. With reference to FIG. 7, the blades 14′ each have a single cutting edge 35 which is tapered to a point with such blades being generally referred to as stab blades.

Regardless of the shape of the blades 14, 14′, each of the blades 14, 14′ is provided with a stem 36, and a tab 38 depending therefrom. The stem 36 and the tab 38 are formed to mountingly engage one of the blind apertures 28, such as being force fit into the blind aperture 28. The stem 36 is formed to be received in the circular portion 32 of the blind aperture 28 in an interference fit, while the tab 38 is formed to be received in the rectangular portion 30 in an interference fit. The orientation of the tab 38 dictates the orientation of the blade 14, 14′. Thus if the tab 38 is aligned vertically, the blade 14, 14′ has a corresponding orientation, whereas, if the tab 38 is aligned horizontally, the blade 14, 14′ has a corresponding orientation. The placement of the tab 38 into the blind aperture automatically aligns the respective blade 14, 14′ via the rectangular portion 30. As can be readily appreciated the locations and spacings of the blind apertures 28 dictate the relative locations and spacings of the blades 14, 14′.

Advantageously, the blades 14, 14′ may be stamped formed, with the stem 36 and the tab 38 being used to hold the respective blade 14, 14′ during a polishing procedure which forms the respective cutting edge(s) 34, 35. It is also preferred that the blades 14, 14′ be coated with diamond-like carbon and lubricious polymer. This coating preserves the sharpness and lubricity of the blades 14, 14′, thereby extending the usefulness of the apparatus 10.

In mounting the blades 14, 14′ to the handle 12, the blades 14, 14′ are spaced apart along a single blade axis 40. The blade axis 40 is oblique to the longitudinal axis 16 and preferably, is perpendicular thereto. In addition, it is preferred that the center axes of the blades 14, 14′ be evenly spaced apart along the blade axis 40 a distance “a”. Preferably, the distance “a” is approximately 3.0 mm. In this manner, evenly spaced hair graft incisions can be made, with sufficient spacing being provided for each hair graft that is to be implanted. In addition, it is preferred that each of the blades 14, 14′ uniformly extend a distance “b” from the distal end 20, with the distance “b” being preferably approximately 5.5 mm from the distal end 20. By limiting the length of hair graft incisions, the incisions can be made having sufficient depth to accept transplanted hair grafts, while preventing blunting of the blades 14, 14′ in contacting bone and also preventing damage to deep vascularity.

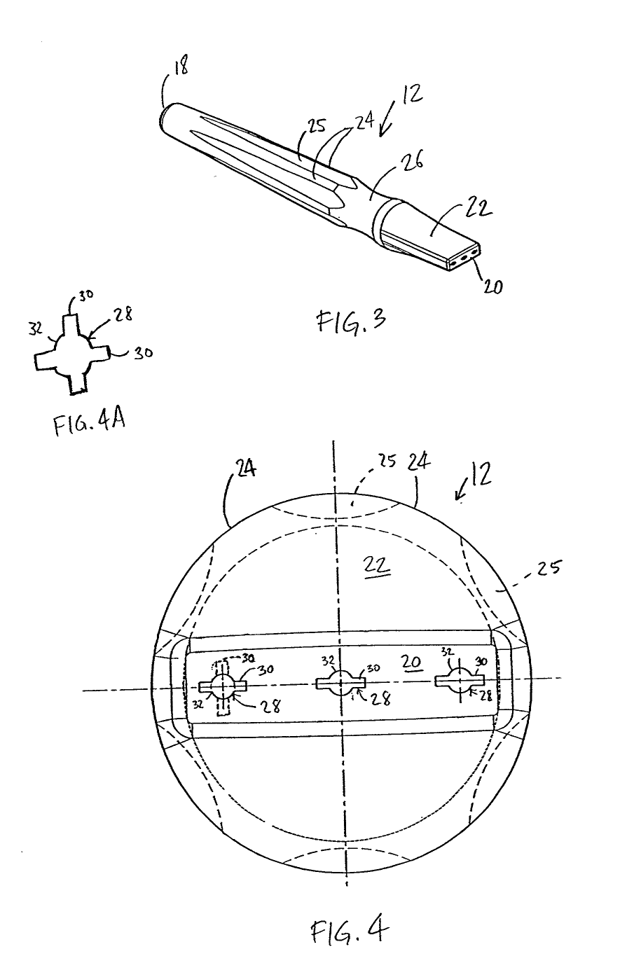

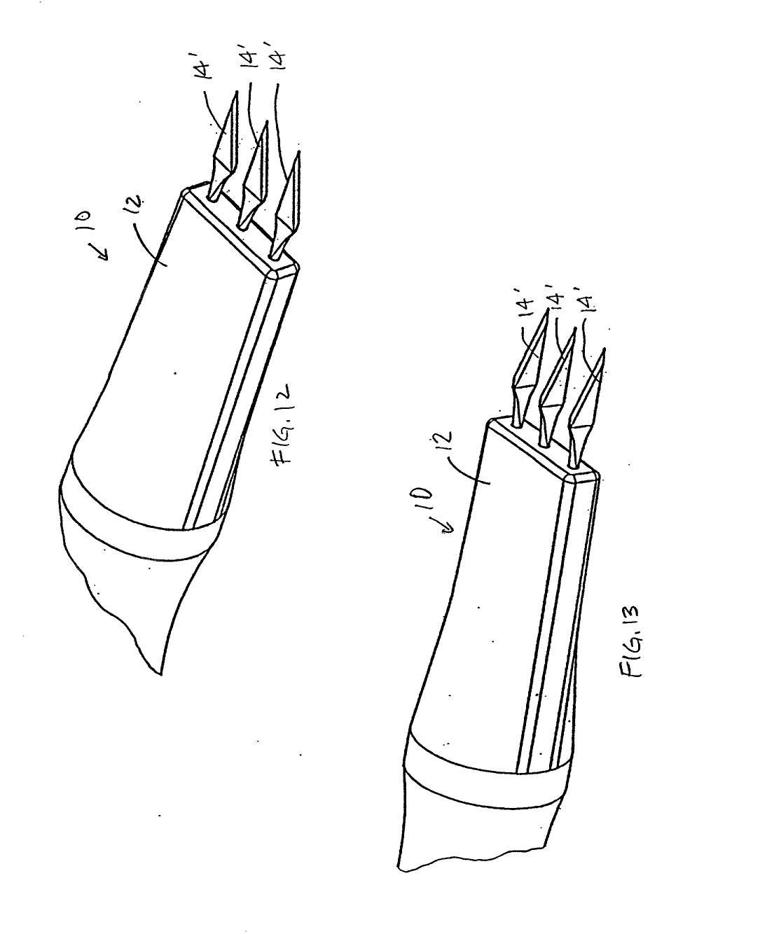

As a further variation, with reference to FIGS. 11 and 13, the blades 14, 14′ may be arranged to be generally parallel and spaced apart along the blade axis 40, in addition to be coplanar as shown in FIGS. 2, 10, and 12. With this variation, the discussion set forth above is fully applicable. The distance “a” continues to be measured between the central axes of the blades 14, 14′. Although other arrangements are possible, horizontally aligned rectangular portions 30 are utilized where the blades 14, 14′ are coplanar, whereas vertically aligned rectangular portions 30 are utilized where the blades 14, 14′ are generally parallel.

With reference to FIG. 8, the apparatus 10 is used to make hair graft incisions 42 in flesh F of a patient. With the apparatus 10, different angular orientations of the hair graft incisions 42 can be achieved, as well as, different depths of incisions 42. For example, a relatively shallow and greatly inclined incision 42′can be formed. In addition, the distal end 20, prevents excessive insertion of the blades 14 into the flesh F and thereby prevents damage to deep vascularity V and contact with bone B. After the hair graft incisions 42 are made, hair grafts 44 can be inserted into the incisions 42. Advantageously, multiple evenly-spaced hair graft incisions 42 can be made with each insertion of the apparatus 10.

It will thus be seen that the objects set forth above, among those made apparent from the preceding description, are efficiently attained and, since certain changes may be made in carrying out the above construction without departing from the spirit and scope of the invention, such as for example more than three blades are utilized, it is intended that all matter contained in the above description and as shown in the accompanying drawings shall be interpreted as illustrative and not in a limiting sense.

It is also to be understood that the following claims are intended to cover all the generic and specific structures of the invention herein described and all statements of the scope of the invention which, as a matter of language might be said to fall therebetween.

What is claimed is:

1. An apparatus for forming hair graft incisions, said apparatus comprising:
an elongated handle having a distal end, said handle extending along a longitudinal axis; and,
a plurality of blades mounted to and extending from said distal end, wherein said blades are arranged on a single blade axis, said blade axis being disposed oblique to said longitudinal axis of said handle.

2. An apparatus as in claim 1, wherein said blades are generally parallel.

3. An apparatus as in claim 1, wherein said blades are generally coplanar.

4. An apparatus as in claim 1, wherein said blades are evenly spaced apart along said blades axis.

5. An apparatus as in claim 4, wherein said blades are evenly spaced approximately 3.0 mm apart.

6. An apparatus as in claim 1, wherein said distal end is generally flat in a plane perpendicular to said longitudinal axis.

7. An apparatus as in claim 1, wherein said blades uniformly extend a same length from said distal end.

8. An apparatus as in claim 7, wherein said same length is predetermined so that during the forming of the hair graft incisions, contact between said blades, on one hand, and bone and deep vascularity underlying the hair graft incisions, on the other hand, is avoided.

9. An apparatus as in claim 7, wherein said same length is approximately 5.5 mm.

10. An apparatus as in claim 1, wherein said blades are stab blades.

11. An apparatus as in claim 1, wherein said blades are spear blades.

12. An apparatus as in claim 1, wherein said blades are each coated with diamond-like carbon and lubricious polymer.

13. An apparatus as in claim 1, wherein said blade axis is disposed generally perpendicularly to said longitudinal axis.

14. A method of transplanting hair comprising the steps of:
preparing hair grafts;
providing an apparatus for forming hair graft incisions, said apparatus having an elongated handle with a distal end, and a plurality of blades mounted to an extending from said distal end;
forming hair graft incisions using said apparatus; and,
inserting said hair grafts into said hair graft incisions.

15. A method as in claim 14, wherein said handle extends along a longitudinal axis, and wherein said blades are arranged along a single blade axis, said blade axis being disposed oblique to said longitudinal axis of said handle.

16. A method as in claim 15, wherein said blade axis is disposed generally perpendicularly to said longitudinal axis.

17. A method as in claim 14, wherein said blades uniformly extend a same length from said distal end.

18. A method as in claim 17, wherein said same length is predetermined so that during the step of forming hair graft incisions, contact between said blades, on one hand, and bond and deep vascularity underlying said hair graft incisions, on the other hand, is avoided.
